(19) United States
(12) Patent Application Publication
(10) Pub. No.: US 2026/0074075 A1
(43) Pub. Date: Mar. 12, 2026

(54) DIAGNOSTIC SYSTEM AND METHOD FOR ASSESSING RISK OF ADVERSE MEDICAL EVENTS FOR ENABLING REDUCTION OF UNPLANNED HEALTHCARE AND/OR MORTALITY

(71) Applicant: Health Navigator Ltd, London (GB)

(72) Inventor: Joachim Werr, Stockholm (SE)

(21) Appl. No.: 19/108,626

(22) PCT Filed: Sep. 6, 2023
(86) PCT No.: PCT/IB2023/058815

§ 371 Date: Mar. 4, 2025

(30) Foreign Application Priority Data
Sep. 6, 2022 (GB) 2213021.5

(51) Int. Cl. G16H 50/30; G16H 10/60; G16H 50/20; G16H 50/70; G16H 80/00

(52) U.S. Cl. CPC G16H 50/30; G16H 10/60; G16H 50/20; G16H 50/70; G16H 80/00

(57) ABSTRACT

Disclosed is a system for assessing risk of adverse medical events leading to unplanned healthcare and/or death, for facilitating mitigation of adverse medical events, system comprising processor(s) configured to: obtain existing healthcare data from data source(s), existing healthcare data comprises accident and emergency data, inpatient data, outpatient data; build predictive model for estimating individual patients'risks for adverse medical events using existing healthcare data and pre-defined set of medical conditions and pre-defined patient characteristics; deploy predictive model for use; obtain first healthcare data, first healthcare data being generated later in time than existing healthcare data; process first healthcare data using predictive model, to predict risk level of adverse medical event(s); identify target set of patients, send communication indicative of target set of patients to data source(s) and/or first device(s) associated with healthcare professional(s), for enabling determination of healthcare intervention(s), healthcare intervention(s) mitigates adverse medical event(s) which reduces mortality of patient.

### TECHNICAL FIELD

The present invention relates to a machine learning model. In particular, though not exclusively, this invention relates to a system and method for assessing a risk of adverse medical events leading to unplanned healthcare and/or death, and for facilitating mitigation of the adverse medical events.

### BACKGROUND

Overcrowding of emergency departments is a major global issue having a detrimental effect on population health and excess load on healthcare infrastructure which ultimately leads to failure of the healthcare infrastructure. A demand of unplanned secondary care is a reason for the overcrowding of the emergency departments. The demand of the unplanned secondar care is continuously increasing and has been further exacerbated owing to absence of in-person services during COVID-19 pandemic. A section of society which mostly needs the unplanned secondary care may include older people, who are living alone and are unbale to manage their chronic health conditions. If not properly managed, their chronic health conditions may get worse, causing fatal consequences for patients. However, such health conditions can be stabilized upon proper identification of patients and their needs, and thereby unplanned emergency hospitalization can be prevented and/or avoided. In a survey, it is found that national Health Service (NHS) of England considers 24% of unplanned hospital admissions, amongst which 40% may be avoidable. Therefore, to target the aforesaid problem, modern analytical tool such as machine learning models has been widely used.

However, conventional machine learning models have several limitations associated with it in terms of accessing and analysis of healthcare data, in terms of performance, and inability in providing proper guidance for effective treatment measures to patients. For example, the conventional machine learning models utilize either an Accident and Emergency (A&E) healthcare data or inpatient healthcare data, leading to limited access to patient's past clinical history. Further, routinely collected healthcare data inherits implicit bias, which leads the conventional machine learning model to exacerbate existing health disparities. The health disparity may arise due to inequalities to health access. The conventional machine learning models are ineffective to reduce the health disparities. Additionally, the conventional machine learning models do not show appropriate discriminatory performance. Moreover, the conventional machine learning models utilize more computationally intensive algorithms such as random forests and support vector machines (SVMs). The aforesaid algorithms are often convoluted and less transparent, therefore may not provide clear and in-depth understanding of the machine learning models to users.

Therefore, in light of the foregoing discussion, there exists a need to overcome the aforementioned drawbacks of conventional machine learning models.

### SUMMARY OF THE INVENTION

A first aspect of the invention provides a system for assessing a risk of adverse medical events leading to unplanned healthcare and/or death, and for facilitating mitigation of the adverse medical events.

The system is enabled to provide a diagnosis of a current health state and consequences like adverse medical events, enabling a determination of a healthcare intervention, i.e. the treatment of the current health state, which leads to an improved health of the patient(s) or at least an end to the degradation or mitigation of the health of the patient(s).

The system comprising at least one processor configured to:
obtain existing healthcare data from at least one data source, wherein the existing healthcare data comprises existing accident and emergency data, existing inpatient data, and existing outpatient data;
build a predictive model for estimating individual patients'risks for adverse medical events leading to unplanned healthcare and/or death, using the existing healthcare data and a pre-defined set of medical conditions and demographics that are likely to lead to the adverse medical events;
deploy the predictive model for use;
obtain first healthcare data from the at least one data source, wherein the first healthcare data comprises first accident and emergency data, first inpatient data, and first outpatient data, the first healthcare data being generated later in time than the existing healthcare data;
process the first healthcare data using the predictive model, to predict a risk level of at least one adverse medical event leading to unplanned healthcare and/or death, for each patient amongst a plurality of patients indicated in the first healthcare data;
identify a target set of patients, wherein each patient belonging to the target set is one whose risk level of the at least one adverse medical event is greater than a first threshold risk level of the at least one adverse medical event;
send a communication indicative of the target set of patients to the at least one data source and/or at least one first device associated with at least one healthcare professional, for enabling determination of at least one healthcare intervention, wherein the at least one healthcare intervention, when provided to said patient, facilitates in at least partially mitigating the at least one adverse medical event which in turn reduces mortality of said patient.

The system is thus providing a diagnosis of a current health state and likely consequences associated with the current health state, the consequences being or comprising the adverse medical events. Through this diagnosis, a healthcare provider or other health service staff is able to prevent further degradation of a health state and the adverse medical effects by being presented with the indication of the set of patients by determining the healthcare intervention.

The term “adverse medical event” refers to a condition of existing disease(s), worsening of the existing disease(s), emergence of new disease(s) in a patient. Notably, the system assesses the risk of the adverse medical events leading to the unplanned healthcare (such as, need of unplanned secondary care, admission to an Accident & emergency (A&E) department, and utilization of other non-elective services) and/or death of the patient. For example, the adverse medical event may be a condition of hypertension in a given patient. The condition of hypertension may lead to Cerebrovascular accident (CVA) resulting in unplanned admission to the Accident & emergency (A&E) department in a hospital.

The term “processor” refers to a computational element that is operable to respond to and process instructions. Furthermore, the term “processor” may refer to one or more individual processors, processing devices and various elements associated with a processing device that may be shared by other processing devices. Such processors, processing devices and elements may be arranged in various architectures for responding to and executing processing steps.

Notably, the at least one processor obtains the existing healthcare data from the at least one data source to enable in building the predictive model to identify patients at an elevated risk of the adverse medical events leading to unplanned healthcare and/or death and for facilitating mitigation of the adverse medical events. Optionally, the system further comprises a data repository, communicably coupled to the at least one processor, wherein the at least one processor is configured to save the existing healthcare data at the data repository.

The term “existing healthcare data” refers to secondary use services (SUS) data. Optionally, the existing healthcare data is pseudonymized healthcare data. Optionally, the existing healthcare data is obtained from at least one of: a healthcare provider, a health system administration, such as Clinical commissioning group (CCG), an insurance organisation, or similar legitimate holder of the existing healthcare data. In one implementation, the existing healthcare data may be provided by the (CCG). The CCG includes a variety of information of the patients. The CCG has at least one of: a number of outpatient attendances, Emergency Department (ED) attendances, inpatient admissions, time and means of the inpatient admissions, conditions with which the patients have been admitted to an inpatient care. Optionally, the existing healthcare data include at least one of: clinically-coded data using International Classification of Diseases (ICD-10), Systematized Nomenclature of Medicine (SNOMED) in Emergency Care Data Set (ECDS), Office of Population Censuses and Surveys (OPCS), Admitted patient care (APC) data set, British National Formulary (BNF).

Notably, the at least one processor obtains the existing healthcare data from the at least one data source for a purpose of training the predictive model. Optionally, the at least one data source is communicably couple to the at least processor. It should be appreciated that, the existing healthcare data used for training the predictive model is high quality and timely (i.e., updated). Optionally, the at least one data source is at least one of: a device associated with a healthcare facility, a device associated with a primary care provider, an out of hours (OOH) service, a device associated with a health trust, an ambulance service, a device associated with a mental health and community facility. In this regard, the existing healthcare data is obtained from various data sources other than associated with aforementioned databases. In an example, the at least one data source may be the device associated with a primary healthcare facility, an urgent treatment center, an alternative healthcare facility, and the like. In another example, the at least one device may be the device associated with a physician, a dentist, a physiotherapist, and the like. Advantageously, the technical effect of obtaining the existing healthcare data from different data sources is that the predictive model is provided with a wide range of training data which results in significant enhancement in predictive performance while predicting the risk level of patients.

The at least one processor obtains the existing healthcare data of a predefined time duration depending upon availability of the existing healthcare data and/or complexity of prediction/estimation required to be performed using the predictive model. As an example, the predefined time duration may be three years. Other values of the predefined time duration are also feasible.

Throughout the present disclosure, the term “predictive model” refers to a machine learning model that is capable of making predictions/estimations. In particular, the predictive model is built to estimate individual patients'risks for the adverse medical events leading to unplanned healthcare and/or death. The predictive model, in use, utilizes the existing healthcare data and the pre-defined set of medical conditions and the pre-defined patient characteristics that are likely to lead to the adverse medical events. In respect of the predictive model, the adverse medical events are outcomes of interest for which estimation of the risk level is to be made. Herein, the phrase “risks for the adverse medical events leading to unplanned healthcare and/or death” encompasses “risk for the adverse medical events” and also “risk for needing unplanned healthcare and/or risk of death”, wherein the risk for needing unplanned healthcare and/or the risk of death depends on the risk for the adverse medical events. The predictive model enables in identifying patients at elevated risk of becoming high-intensity users of emergency and non-elective services. The phrase “high-intensity users” refers to users whose existing medical conditions are worsening and are at risk of being admitted to the hospital. The predictive model is implemented via a software application that performs automated screening of healthcare data (such as the first healthcare data, the second healthcare data, and similar) and enables in identifying high-risk patients.

Optionally, the pre-defined set of medical conditions are pre-generated by at least one healthcare professional. Moreover, optionally, the pre-defined patient characteristics that are likely to lead to the adverse medical events are pre-specified by the at least one healthcare professional. Optionally, the pre-defined set of medical conditions and the pre-defined patient characteristics are generated following prior deployment of the predictive model. In regard to the above, at least one predictive algorithm utilized by the predictive model uses a development strategy that is curated through technical and clinical expertise of the at least one healthcare professional. As an example, the pre-defined set of medical conditions may include a plurality of medical conditions (for example, such as chronic obstructive pulmonary disease (COPD), peripheral vascular disease, asthma, diabetes, Cerebral ischemia and similar) that, in experience of the at least one healthcare professional, are likely to lead to the adverse medical events. As another example, the pre-defined patient characteristics that are likely to lead to the adverse medical events may be an age of 60 years and above, a given gender (for example, such as males), an ethnicity (for example, Asians), and similar.

Optionally, upon building the predictive model, the existing healthcare data is obtained on at least one of: instantly, regular basis, hourly basis, daily basis, a monthly basis, bi-monthly basis, half-yearly basis, yearly basis. In this regard, the existing healthcare data is obtained to enable re-training of the predictive model. As an example, the at least one processor may obtain the existing healthcare data at a time interval of twenty-four hours.

Optionally, when building the predictive model, the at least one processor is configured to:
normalize the existing healthcare data into a unified feature set, wherein the unified feature set comprises features pertaining to patient characteristics, medical diagnosis, and healthcare provider activity;
execute data quality and imputation checks on the unified feature set;
train and validate the predictive model using a first portion of the unified feature set and at least one machine learning algorithm to build weights against each medical condition in the pre-defined set of medical conditions; and
test the predictive model that is trained, using a second portion of the unified feature set.

In this regard, the normalization of the existing healthcare data into the unified feature set is performed using a flexible feature normalization layer built in a database system (such as PostgreSQL, or similar) that maintains the existing healthcare data. The existing healthcare data may include clinically-coded data using International Classification of Diseases (ICD)-10, Systematized Nomenclature of Medicine (SNOMED) (in Emergency Care Data Set (ECDS)) and OPCS as well as widely used national datasets such as APC, ECDS and the British National Formulary (BNF), thus such normalization is important so that the existing healthcare data can be utilized properly for building the predictive model. Optionally, the existing healthcare data is pseudonymized acute data. The unified feature set comprises features extracted from the existing healthcare data, wherein said features pertain to patient characteristics, medical diagnosis, and healthcare provider activity. Optionally, the unified feature set further comprises biomarkers. Examples of the biomarkers include, but are not limited to, a resting heart rate of over 110 beats per minute, a blood sugar level greater than 140 mg/dl, sleep apnea, and similar. These different categories of features are non-limiting, and more categories of features could be utilized as well. The data quality and imputation checks are executed (for example, by a processing layer) on the unified feature set to ensure that data formats and values within the unified feature set are within an expected range (i.e., a realistically feasible range) for use in building the predictive model. In this regard, features of the unified feature set that fail the data quality and imputation checks may be discarded. Optionally, the at least one machine learning algorithm is at least one predictive algorithm. It will be appreciated that training, validation and testing of the predictive model are well-known steps for building machine learning-based models, which when employed, enable the predictive model to be highly accurate in making estimations/predictions.

Optionally, the first portion of the unified feature set is 65% to 85% of an entirety of the unified feature set. As an example, the first portion of the unified feature set may be 75% of the entirety of the unified feature set. Optionally, the second portion of the unified feature set is 15% to 35% of the entirety of the unified feature set. As an example, the second portion of the unified feature set may be 25% of the entirety of the unified feature set. The term “weight” used herein refers to a measure of an ability of a factor (i.e., a given medical condition) to influence deductions made by the predictive model. Notably, the weights are built against each medical conditions in the pre-defined set of medical conditions. The phrase “build weights against each medical condition” means that the at least one machine learning algorithm determines the weights that are to be assigned to each medical condition in the pre-defined set of medical conditions.

Optionally, the pre-defined set of medical conditions are determined by at least one healthcare professional based upon the adversity of the medical conditions. As an example, the pre-defined set of medical conditions may be distributed across approximately sixty-five, chronic, acute and avoidable medical conditions. Optionally, the pre-defined set of medical conditions is a subset of the unified feature set. Optionally, in this regard, weights may also be built against other subsets of the unified feature set. For example, the weights may be built against biomarkers, patient characteristics and the like.

The weights are built by analyzing the unified feature set to determine how much each subset of the unified feature set impacts the risk for adverse medical events leading to unplanned healthcare and/or death. Optionally, the weights lie in a range of 0 to 1. Alternatively, optionally, the weights lie in a range of −1 to +1. Yet alternatively, optionally, the weights lie in a range of 0 to 100. Other ranges for weights are also feasible. It will be appreciated that each subset of the unified feature set may differently impact the risk for adverse medical events leading to unplanned healthcare and/or death, and thus weights assigned to the pre-defined set of medical conditions would be different. As an example, weight built against COPD may be 0.8 at a place where weather conditions are harsh and 0.75 at the place where the weather conditions are conducive. As another example, weights built against COPD and anemia may be 0.8 and 0.5, respectively, as anemia is relatively less likely to impact the risk for adverse medical events leading to unplanned healthcare and/or death as compared to COPD.

Optionally, the at least one processor is further configured to share test results of testing the predictive model, to the at least one first device and/or the at least one data source. These test results are indicative of accuracy of the predictive model, and are shared to the at least one first device and/or the at least one data source so that users thereof are made aware of performance of the predictive model. Optionally, the test results may include values of at least one testing parameter, the at least one testing parameter being at least one of: discriminatory performance, sensitivity, specificity. Notably, the “discriminatory performance” is measured as Area Under a Curve (AUC), the “sensitivity” is a ratio of a number of correctly-identified patients in a test target set to a total number of patients that actually belong to the test target set, and the “specificity” is a ratio of a number of correctly-identified patients not belonging to the test target set to a total number of patients that actually do not belong to the test target set.

Optionally, the weights are built against each medical condition in the pre-defined set of medical conditions using pre-defined weights. In this regard, pre-defined weights may be assigned to the medical conditions, and said weights may be adjusted by the at least one machine learning algorithm. When a given pre-defined weight assigned to a given medical condition does not require adjusting, a given weight built against the given medical condition is same as the given pre-defined weight. In some implementations, the pre-defined weights can be same as weights used in past deployments of the predictive model. In some other implementations, the pre-defined weights can be tailored specifically by a system administrator of the system. In such implementations, the tailoring of the pre-defined weights is done specific to a healthcare provider or a healthcare system for which the predictive model is deployed. Optionally, the tailoring of the pre-defined weights is done using a portion of the existing healthcare data that is provided by the healthcare provider or the healthcare system.

Optionally, the predictive model is trained and validated using k-fold cross validation, and wherein prior to testing the predictive model that is trained, the at least one processor is further configured to:
generate model evaluation scores for the k folds; and
fine tune the predictive model by adjusting the weights, based on the model evaluation scores, for improving an accuracy of the predictive model.

In this regard, prior to the k-fold cross validation, a Lasso and elastic-net regularized linear model (for example, such as GLMnet) is fitted to the first portion of the unified feature set (i.e., training data). The k-fold cross validation is a technique that is used to estimate skill of the predictive model on new data (i.e., data that is different from training data. In k-fold cross validation, k iterations of training and validation are performed wherein at each iteration—the first portion of the unified feature set is shuffled and split into the k folds which are equally-sized; and then k−1 folds are used to train the predictive model while 1 remaining fold is used to evaluate (i.e., validate) the training performed using the k−1 folds. In this way, each fold is used k−1 times to train the predictive model and 1 time to validate the predictive model. In an example, k may be equal to 10. Other values of k are also feasible. A model evaluation score is generated at each of the k iterations, and is stored (for example, at the data repository communicably coupled with the at least one processor). The model evaluation scores are indicative of how well the predictive model is learning to make estimations/predictions. Optionally, the model evaluation scores are determined as at least one of: a number of false identifications of patients as belonging to the target set, a number of false mis-identifications of patients as not belonging to the target set, a number of true identifications of patients belonging to the target set, a number true identification of patients not belonging to the target set. When fine tuning the predictive model, different weights may be adjusted differently, for example by increasing their value, decreasing their value, or by maintaining their value. The fine tuning of the predictive model is intended to improve the accuracy of the predictive model, and when performed, leads to highly accurate results at the time of testing the predictive model that is trained. Moreover, hyperparameters and weights of the predictive model are optimized using the k-fold cross validation to avoid statistical overfitting.

Optionally, the patient characteristics comprise one or more of: age, gender, ethnicity, social and economic deprivation, and wherein the at least one processor is further configured to:
identify a set of vulnerable patients, based on the patient characteristics, wherein a vulnerable patient is one who belongs to one or more of: a vulnerable age group, a vulnerable gender, a vulnerable ethnicity, a deprived group;
determine a vulnerability score for each patient in the set of vulnerable patients, based on data associated with patient characteristics of said patient; and
enhance the risk level of the at least one adverse medical event for each vulnerable patient having a vulnerability score higher than a threshold vulnerability score, by a predetermined level.

The “patient characteristics” are attributes associated with patients, wherein such attributes may be indicative of health conditions of the patients. The term “social and economic deprivation” refers to social and economic attributes that are indicative of a state of deprivation of the patients. Patients in the state of deprivation are less likely to be in good health and/or to have access to basic healthcare and specialized healthcare. Examples of vulnerable age groups include, but are not limited to, 0-6 months, 0-12 months, 1-3 years, 50-60 years, and 60 years and above. Examples of vulnerable genders may be male, polygender, and the like. Examples of vulnerable ethnicities may include, but are not limited to, Asians, Blacks or African Americans, and Hispanic or Latino. Examples of deprived groups may be people who live below poverty line, people who are unemployed, people who live in unsanitary dwellings, people who are homeless, and the like.

Optionally, the set of vulnerable patients is identified by processing the data associated with the patient characteristics. In this regard, the at least one processor may compare data associated with the patient characteristics of each patient against a pre-defined reference data of vulnerable patients, and when the data associated with the patient characteristics of a patient matches with the pre-defined reference data of vulnerable patients, the at least one processor identifies said patient to belong to the set of vulnerable patients. The pre-defined reference data of vulnerable patients may be provided by the system administrator of the system and/or a user of the at least one first device and/or the at least one data source.

The “vulnerability score” of a given vulnerable patient is a measure of how vulnerable the given vulnerable patient is to the at least one adverse medical event. The vulnerability score may be calculated on any suitable scale, for example, 0 to 1, 0 to 10, 0 to 100, and similar. Higher the vulnerability score, greater is the measure of vulnerability of the given vulnerable patient to the at least one adverse medical event, and vice versa. Optionally, the vulnerability score for each patient is determined using at least one of: an extent of matching between the data associated with the patient characteristics of said patient with the pre-defined reference data of vulnerable patients, a lookup table including vulnerability scores corresponding to data values of the data associated with patient characteristics, an Index of Multiple Deprivation (IMD). In one implementation, the vulnerability score for each patient may be determined using the IMD.

It will be appreciated that patients belonging to the set of vulnerable patients are at a higher risk of the at least one adverse medical event, as compared to patients that do not belong to the set of vulnerable patients. Moreover, vulnerable patients having vulnerability scores higher than the threshold vulnerability score are highly prone to the at least one adverse medical event in future. Therefore, for such vulnerable patients, the risk level of the at least one adverse medical event is enhanced by the predetermined level, to account for such future risk. This also ensures that such vulnerable patients get included in the target set, so that their medical analysis is performed by healthcare professionals for targeting healthcare intervention(s). The threshold vulnerability score may be pre-determined (for example, pre-set by the at least one processor), may be determined to be an average of vulnerability scores for the patients in the set of vulnerable patients, and similar.

Notably, the at least one processor further deploys the predictive model to be used by the system administrator of the system. Optionally, the at least one processor deploys the predictive model to the at least one data source. Additionally, optionally, the at least one processor deploys the predictive model to the at least one first device associated with the at least one healthcare professional. Optionally, the at least one device is implemented as at least one of: a smartphone, a smartwatch, a tablet computer, a laptop computer, a desktop computer, an infotainment device, and a personal digital assistant. Optionally, the healthcare professional is at least one of: a primary healthcare provider, a secondary healthcare provider, an ambulance driver, a hospital staff member. Optionally, the at least one first device is a data source.

Notably, upon building the predictive model, the at least one processor obtains the first healthcare data from the at least one data source. Optionally, the first healthcare data is obtained from the at least one first device associated with the at least one healthcare professional. Additionally, optionally, the at least one processor saves the first healthcare data in the data repository. The first healthcare data includes information of patients admitted to at least one of: the A&E department, the inpatient department, the outpatient department. The first healthcare data to be used in the predictive model for risk scoring is generated later in time than the existing healthcare data. For example, the first healthcare data to be used in the predictive model may be generated in subsequent 30 days of the existing healthcare data. Optionally, the at least one processor stores the first healthcare data at the data repository. It should be noted that the first healthcare data before being used in the predictive model is normalized into the unified feature set and undergoes data quality and imputation checks as already described above. Optionally, the first healthcare data is obtained on at least one of: a daily basis, hourly basis, weekly basis. In one implementation, the at least one processor obtains the first healthcare data within the time interval of twenty-four hours to generate the outcome of interest for each patient belonging to the first healthcare data.

Upon receiving the first healthcare data, the predictive model predicts the risk level of the at least one adverse medical of the patients'indicated in the first healthcare data. Optionally, the risk level is calculated in terms of probability of achieving the outcome of interest for each patient belonging to the first healthcare data. In this regard, the predictive model utilizes the existing inpatient data, the existing A&E data, and the existing outpatient data along with the first healthcare data to calculate the probability of achieving the outcome of interest for each patient belonging to the first healthcare data. For example, the predictive model estimate may estimate the probability of each patient indicated in the first healthcare data spending three or more unplanned bed days in hospital in subsequent six months.

Notably, upon predicting the risk level of each patient, the predictive model identifies the target set of patients. In this regard, the patients belonging to the target set are those patients whose risk level of the at least one adverse medical event is greater than the first threshold risk level of the at least one adverse medical event. For example, a patient suffering from a condition of high level of cholesterol may be at a high risk of heart attack than a person having a normal level of the cholesterol. Therefore, the target set of patients may comprise the patient having the high level of cholesterol. Optionally, the at least one processor stores the target set of patients at the data repository. It will be appreciated that first threshold risk levels of different adverse medical events are different. In this regard, the first threshold risk level of a given adverse medical event may depend upon fatality of the adverse medical event. As an example, the condition of heat attack may be more fatal than a condition of bone fracture. In such a case, the first threshold risk level of the heart attack may be less then the first threshold risk level of the bone fracture.

Notably, upon determination of the target set of patients, the at least one processor sends the communication indicative of the target set of patients to the at least one data source and/or the at least one first device associated with the at least one healthcare professional. The at least one processor determines the at least one healthcare intervention for each patient belonging to the target set. Optionally, determination of the at least one healthcare intervention could be made manually, semi-automatically, or fully automatically. Optionally, the at least one healthcare professional can choose when and how to implement healthcare interventions proposed by the at least one processor. Optionally, the at least one healthcare intervention comprises initial assessment to develop a personalized care plan for each patient belonging to the target set followed by telephonic communication for management of the healthcare intervention. The telephonic communication may be conducted by a healthcare professional. Optionally, a communication with each patient is conducted on video calls. During this communication, each patient receives at least one of: motivational conversations, support for self-care, educational conversations targeting the adverse medical event, coordination of social and medical services. Notably, the at least one healthcare intervention targets the adverse medical event of each patient belonging to the target set which in turn reduces the mortality of the said patient. The reduction in the mortality of each patient following the at least one healthcare invention is described in an experimental data provided below.

The present disclosure provides the aforementioned system, for assessing the risk of the adverse medical events leading to unplanned healthcare and/or death, and for facilitating mitigation of the adverse medical events. The system utilizes wide range of the existing healthcare data including the existing accident and emergency data, the existing inpatient data, and the existing outpatient data. Utilization of the aforesaid existing healthcare data of different departments results in granular representation of patient's clinical and activity history as well as personal characteristics. The predictive model enables healthcare providers to target appropriate cohorts of patients (i.e., the target set of patients) at the elevated risk of the adverse medical events leading to unplanned healthcare and/or death and provides the at least one healthcare intervention that is scalable and can be standardized across healthcare infrastructures. The at least one healthcare intervention provided by the system is customized depending upon targeted medical condition of each patient. Further, the predictive model significantly reduces cost and allows for a long-term planning of the secondary care required by the target set of patients. Further, the predictive model of the present invention shows the discriminatory performance of 0.8, the sensitivity of 0.8, and the specificity of 0.65. Moreover, the at least one healthcare intervention provided by the system results in significant reduction in the mortality the patients.

Optionally, the at least one first device is configured to receive a plurality of first inputs provided by the at least one healthcare professional, the plurality of first inputs pertaining to a selection of a first subset of patients from amongst the target set such that a portion of the first healthcare data that is associated with each patient selected to belong to the first subset complies with at least one inclusion criteria.

In this regard, upon receiving the target set of patients on the at least one device, the at least one healthcare professional further narrows down the target set of patients to obtain the first subset of patients. Optionally, the at least one processor stores the first subset of patients to the data repository. Optionally, the inputs are in the form of at least one of: a click input, a touch input. Optionally, the target set of patients are reviewed by the at least one healthcare professional to ascertain each patient's suitability to the at least one healthcare intervention, using at least one of: own professional judgement, patient's electronic health record on the at least one first device.

Optionally, the at least one inclusion criteria is at least one of: a given patient being at least eighteen years of age, a given patient having at least one emergency attendance in a predefined duration, a given patient whose risk level of the at least one adverse and potentially amenable medical event is greater than a second threshold risk level of the at least one adverse and potentially amenable medical event wherein the second threshold risk level is higher than the first threshold risk level. Optionally, a magnitude of the second threshold risk level is more than the first threshold risk level. In one implementation, a given patient of less than eighteen years of age may not be included in the first subset of patients. In another implementation, a given patient who has not been admitted to the A&E department may not be included in the first subset of patients. In yet another implementation, a given patient whose risk level of the adverse medical event is less than the second threshold value, may not be included in the first subset of patients. Advantageously, the first subset of patients is generated to ensure that the patients need the at least one healthcare intervention and/or are in a state of adequately receiving at least one healthcare intervention leading to maximum benefit of the at least one healthcare intervention.

Optionally, the at least one first device is further configured to receive a plurality of second inputs provided by the at least one healthcare professional, the plurality of second inputs pertaining to a selection of a second subset of patients from amongst the first subset of patients such that the portion of the first healthcare data that is associated with each patient selected to belong to the second subset is non-compliant with at least one exclusion criteria. In this regard, the healthcare professional further analyzes the suitability of patients belonging to the first subset of patients for receiving the at least one healthcare intervention. Depending upon suitability of the patients, the at least one healthcare professional further narrows down the first subset of patients to generate the second subset of patients.

Optionally, the exclusion criteria is at least one of: a given patient who have had contact with the healthcare provider for a predefined diseases, a given patient having an estimated life expectancy less than a year, a given patient undergone a major surgery in past six months, a given patient having a planned surgery, a given patient having severe hearing loss, a given patient having lingual limitation, a given patient having less cognitive ability to receive and/or respond to telephonic communication, a given patient having no telephonic connection, a given patient who is pregnant. Examples of the predefined disease include, Dementia, Psychotic disorders, Mental disorders caused by drug misuse, Terminal cancer, and the like. It will be appreciated that the aforesaid exclusion criteria are exemplary. Other exclusion criteria are well within the scope of the present invention. Advantageously, the second subset of patients is generated to ensure that the patients belonging to the second subset are suitable to receive the at least one healthcare intervention, resulting in maximum benefit of the at least one healthcare intervention.

Optionally, the at least one processor is further configured to:
receive, from the at least one data source and/or the at least one first device, feedback pertaining to accuracy of the target set of patients and suitability of the target set of patients to receive the at least one healthcare intervention;
determine a performance metric of the predictive model, based on the feedback; and
communicate the feedback and the performance metric to the at least one processor, for initiating re-training of the predictive model based on the feedback and the performance metric.

In this regard, the target set of patients identified by the predictive model are reviewed by the at least one healthcare professional. Optionally, the feedback pertaining to the accuracy of the target set of patients is estimated based on the suitability of the patients belonging to the target set to receive the at least one healthcare intervention. Optionally, the suitability of the patients is checked based on the inclusion criteria and the exclusion criteria as described above. Optionally, the at least one processor monitors the performance of the model (i.e., determines suitability of the target set of patients) as well as variation in a raw data provided from the at least one data source.

Further, the at least one processor also determines the performance metric of the predictive model based on the feedback received from the at least one data source and/or the at least one first device. Optionally, the performance metric is at least one of: an Area Under the ROC Curve (AUC-ROC), a Concordant-Discordant Ratio, Cross-Validation, Gain and Lift Chart, Kolmogorov Smirnov chart, Mean Square Error. Optionally, determination of the performance metric of the predictive model could be made manually, semi-automatically, or fully automatically. In one implementation, the performance metric may be preset manually. As an example, the AUC-ROC may be preset at 0.8, a false negative rate may be preset at 0.15 or lower, and the sensitivity may be preset at more than 0.7. Lastly, depending upon magnitude of the feedback and the performance metric, the at least one processor initiates retraining of the predictive model using the existing healthcare data to achieve accurate selection of the patients in the target set. In this regard, the at least one processor initiates re-training of the predictive model, if the magnitude of the feedback and the performance matric are less than a given threshold. Advantageously, the technical effect of receiving the feedback is that in addition to the performance metric, the feedback provides an additional check for measuring accuracy of the predictive model leading to accurate analysis of performance of the predictive model resulting in enhanced outcome of the predictive model.

Optionally, the at least one processor is further configured to receive, for each patient belonging to the target set, an input indicative of the at least one healthcare intervention, from the at least one data source and/or the at least one first device. In this regard, upon determination of the target set of patients, the at least one processor receives the at least one healthcare invention for each patient belonging to the target set. Optionally, the at least one healthcare invention is received depending upon the medical condition, the patient in the target set is suffering. Optionally, the at least one processor stores the at least one healthcare intervention at the data repository. Additionally, optionally, the at least one processor is further configured to perform evaluation of the at least one healthcare intervention against a pre-determined dataset from the at least one data source.

Optionally, the at least one processor is further configured to process the existing healthcare data to extract an additional feature set, wherein the additional feature set comprises time-dependent variables indicative of patient condition and activity, wherein when training the predictive model, the at least one processor is configured to also use the additional feature set. Optionally, time-dependent variables capture at least one of: a transiency of journey of a given patient, a change in hospital activity over time when assessing the risk level of the adverse medical impact of the given patient. Examples of the transiency of the journey include information regarding how the given patient interacted with hospital, attendance in the outpatient department, and the like. Additionally, optionally, the time-dependent variables capture a number of patients admitted in the hospital, a medical condition with which the patients were admitted to the hospital. As an example, the time-dependent variables include the number of patients attended the A&E department in last 30, 90, and 180 days. As another example, the time-dependent variable may be the number of patients admitted in the hospital with Chronic obstructive pulmonary disease (COPD) in the last 30, 90, and 180 days. Optionally, the time-dependent variables are extracted from the existing healthcare data using a R script. Advantageously, the technical effect of using the additional feature set is that the additional feature comprising the time-dependent variables very well capture the patient's conditions and the previous activity leading to adequate training of the predictive model resulting in enhanced accuracy while predicting the risk level of at least one adverse medical event leading to unplanned healthcare and/or death.

Optionally, the at least one processor is further configured to:
obtain second healthcare data from the at least one data source, wherein the second healthcare data comprises second accident and emergency data, second inpatient data, and second outpatient data, the second healthcare data being generated later in time than the first healthcare data;
process the second healthcare data to predict an updated risk level of the at least one adverse medical event for: each patient not belonging to the target set, each patient belonging to the target set who received the at least one healthcare intervention;
update the target set of patients, based on the updated risk levels; and
send a communication indicative of the updated target set of patients, to the at least one data source and/or the at least one first device.

In this regard, the second healthcare data comprises the information of patients admitted to the A&E department, the inpatient department, the outpatient department. Notably, the second healthcare data is generated later then the first healthcare data. A magnitude of the updated risk level of the at least one adverse medical event obtained using the second healthcare data may be greater than or equal to the risk level obtained using the first healthcare data. Optionally, the updated risk level is predicted for the patients who were previously identified as low risk, and hence were not included in the target set. Additionally, optionally, the updated risk level is predicted for patients belonging to at least one of: the target set, the first subset and the second subset. The updated risk level is predicted to ensure that the patients who were previously identified as the low risk and/or received the at least one healthcare intervention may have felt deterioration in their health condition, may be admitted to the hospital in the A&E department, in the inpatient department, the outpatient department in subsequent days, can be accurately identified based on the second healthcare data by the predictive model. Optionally, the updated target set of patients who are identified using the second healthcare data have equal or a greater number of patients than the patients identified using the first healthcare data. Advantageously, the technical effect of using the second healthcare data is that the patients who were previously not identified in the target set and/or the patients who have received the at least one healthcare invention are not excluded out of the existing healthcare data, and they can be re-identified if their condition deteriorates and subsequent visits to the hospital increases. Owing to the above, accuracy of the predictive model is enhanced significantly, and the mortality of the patients have reduced significantly.

Optionally, the at least one processor is further configured to:
deploy a pseudonymization engine at the at least one data source, wherein the pseudonymization engine employs a hashing algorithm to pseudonymize a given healthcare data;
deploy communication interfaces connecting the at least one data source with at least the at least one processor and the at least one first device to enable at least one of: sending of real-time updates of the given healthcare data, sending of the given healthcare data only upon pseudonymization, re-identification of the given healthcare data of a subset of patients within the target set of patients at a target device wherein the subset of patients includes patients with risk levels greater than a threshold high-risk level of the at least one adverse medical event.

In this regard, the given healthcare data (i.e., the existing healthcare data, the first healthcare data, the second healthcare data, and similar) is pseudonymized for Information Governance (IG) and Compliance. The term “pseudonymize” refers to a data management and de-identification technique using which personally identifiable information the given healthcare data is replaced by pseudonyms (i.e., artificial identifiers). Optionally, the pseudonymization engine comprises a de-identification engine configured to perform pseudonymization of data and a re-identification engine configured to perform re-identification of pseudonymized data. The de-identification engine employs the hashing algorithm for pseudonymizing the given healthcare data. In an example, the pseudonymization engine may be deployed at the at least one data source using a LiteConnector written in the. NET framework using C # and Visual Basic. Optionally, a database at which the given healthcare data is maintained by the at least one data source is a lightweight database (rather than flat files). This improves speed of pseudonymization and communication of data. Optionally, the communication interfaces connecting the at least one data source with at least the at least one processor and the at least one first device are implemented as webhooks. Optionally, the real-time updates of the given healthcare data are sent as soon as they are available at the at least one data source. When the given healthcare data is sent only upon pseudonymization, it ensures compliance of the system with data privacy requirements and regulations. When the re-identification of the given healthcare data of the subset of patients within the target set of patients is enabled, it enables healthcare professional(s) associated with the target device to screen patients appropriately and make correct decisions (for example, of which healthcare interventions would suit such patients).

Optionally, when a same patient identifier is used for referring to a given patient in different healthcare data, the hashing algorithm creates a same hashed identifier for the given patient across the different healthcare data. This allows for linkage of patient information across different types of data sources, even upon pseudonymization of data. This in turn leads to better diagnosis and decision making with respect to the given patient as healthcare professional(s) screening such a patient's data would have access to an entirety of healthcare data associated with the given patient.

As mentioned above, the system provides a diagnosis of a current health state and enables a healthcare provider or other health service staff to determine a healthcare intervention, and this diagnosis is improved compared to prior art diagnostic systems.

Optionally, the at least one adverse medical event is at least one of: unplanned hospitalization, unplanned outpatient visit, requirement of emergency services, readmission to a healthcare facility, stranding in a healthcare facility, serious fall, frailty progression, worsening of existing medical conditions, emergence of new medical conditions, pediatric response exacerbation, high-intensity primary care usage. In this regard, the at least one adverse medical event is a medical condition leading to a need of instant secondary healthcare for the patients, if not provided leading to fatal consequences. In one example, the at least one adverse medical event may be the unplanned hospitalization for various conditions such as Cerebral ischaemia (stroke), peripheral vascular disease, fracture and the like. In a second example, the at least one adverse medical event may be the unplanned outpatient visits for various conditions such as chest pain due to Stable Ischemic heart disease, asthma, chromic sinusitis, hypertension, and the like. In a third example, the at least one adverse medical event may be the requirement of emergency services such as an ambulance. In a fourth example, the at least one adverse medical event may be the condition of the frailty progression in older patients following an appointment with the primary care provider.

A second aspect of the present invention provides a method for assessing a risk of adverse medical events leading to unplanned healthcare and/or death, and for facilitating mitigation of the adverse medical events, the method comprising:
obtaining existing healthcare data from at least one data source, wherein the existing healthcare data comprises existing accident and emergency data, existing inpatient data, and existing outpatient data;
building a predictive model for estimating individual patients'risks for adverse medical events leading to unplanned healthcare and/or death, using the existing healthcare data and a pre-defined set of medical conditions and pre-defined patient characteristics that are likely to lead to the adverse medical events;
deploying the predictive model for use;
obtaining first healthcare data from the at least one data source, wherein the first healthcare data comprises first accident and emergency data, first inpatient data, and first outpatient data, the first healthcare data being generated later in time than the existing healthcare data;
processing the first healthcare data using the predictive model, for predicting a risk level of at least one adverse medical event leading to unplanned healthcare and/or death, for each patient amongst a plurality of patients indicated in the first healthcare data;
identifying a target set of patients, wherein each patient belonging to the target set is one whose risk level of the at least one adverse medical event is greater than a threshold risk level of the at least one adverse medical event; and
sending a communication indicative of the target set of patients to the at least one data source and/or at least one first device associated with at least one healthcare professional, for enabling determination of at least one healthcare intervention, wherein the at least one healthcare intervention, when provided to said patient, facilitates in at least partially mitigating the at least one adverse medical event which in turn reduces mortality of said patient.

The method steps for assessing the risk of adverse medical events leading to unplanned healthcare and/or death, and for facilitating mitigation of the adverse medical events, are already described above. Advantageously, the aforesaid method is easy to implement, provides fast results, and does not require expensive equipment. And, an improved diagnosis is provided which enables for determining intervention steps to be taken to prevent a health care stat to deteriorate, thus healing or curing the current health state and any adverse medical effects of the health state.

A third aspect of the present invention provides a computer program product comprising a non-transitory machine-readable data storage medium having stored thereon program instructions that, when accessed by at least one processor, cause the at least one processor to implement the method of the second aspect.

The term “computer program product” refers to a software product comprising program instructions that are recorded on the non-transitory machine-readable data storage medium, wherein the software product is executable upon a computing hardware for implementing the aforementioned steps of the method for classification and/or prediction on unbalanced datasets.

In an embodiment, the non-transitory machine-readable date storage medium can direct a machine (such as computer, other programmable data processing apparatus, or other devices) to function in a particular manner, such that the program instructions stored in the non-transitory machine-readable data storage medium cause a series of steps to implement the function specified in a flowchart corresponding to the instructions. Examples of the non-transitory machine-readable data storage medium includes, but is not limited to, an electronic storage device, a magnetic storage device, an optical storage device, an electromagnetic storage device, a semiconductor storage device, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, or any suitable combination thereof.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### Experimental Data for the Predictive Model

A randomized control trial (RCT) was performed for identification of the patients at the elevated risk of the adverse medical events leading to the unplanned healthcare and/or death. The predictive model was trained on three years of local hospital data as the existing healthcare data. The local hospital data was pseudonymized and focused on the A&E department, the inpatient department and the outpatient department of a local hospital. The Lasso and elastic-net regularized linear model (for example, such as GLMnet) was fitted to the existing healthcare data. The GLMnet was shown to perform just as well as boosted approaches (e.g., XG Boost and Adaboost), but was chosen for its transparency and intelligibility. The model was internally validated using the k-fold cross-validation and a split-sample approach. The predictive model showed a discriminatory performance of 0.8 on a test data (i.e., the first healthcare data, the sensitivity of 0.8, and the specificity of 0.65, across all sites included in the trial. Moreover, the at least one healthcare intervention provided by the system results in significant reduction in the mortality of the patients.

The system thus provides a diagnosis of a current health state and consequences like adverse medical events, enabling a determination of a healthcare intervention, i.e. the treatment of the current health state, which leads to an improved health of the patient(s) or at least an end to the degradation or mitigation of the health of the patient(s).

### Experimental Data for the Mortality of the Patients Using the Predictive Model

The patients belonging to the second subset were subjected to a study to determine an impact of the at least one healthcare intervention on the mortality of the patients. The study was conducted by randomized control trial of the patients. The patients were randomized using an online random sequence generator into two groups, namely an intervention group, a control group. The patients were randomized in the intervention group and the control group in a ratio of 2:1 respectively. Patients in the intervention were provided the at least one healthcare invention and the patients in the control group were provided usual care. It should be noted that, a follow-up observation period of two-years from the point of randomization was included for evaluating the mortality of the patients. The patients were distributed across different categories such as age, gender, and Index of Multiple Deprivation (IMD) deciles to calculate the mortality using a Kaplan-Meier curve. The age was divided into different categories, such as under the age of 65 years, from 65 years to 75 years, from 75 years to 85 years and 85 years and above. The IMD deciles were divided into two categories, a first category of 50% most deprived groups (an IMD decile less than or equal to 5) and 50% least deprived groups (the IMD decile greater than 5). Over the two-year follow-up observation period, an overall 18% decrease in the mortality of the patients belonging to the intervention group was obtained as compared to the patients belonging to the control group. The reduction in mortality was calculated in terms of Hazard ratio (HR). The “Hazard ratio” is a measure of how often a particular event happens in one group compared to how often it happens in another group, over a predefined time period. The patients in the intervention group showed a lower overall mortality than the patients in the control group. The HR of the patients in the intervention group was 0.81 at 95% confidence interval (CI) equivalent to 0.61, 1.07. Further, amongst the patients in the intervention, male patients showed a large reduction in the mortality as compared to female patients. The HR of the male patients was 0.74 at 95% CI equivalent to 0.51, 1.05, whereas the HR of the female patients was 0.95 at 95% CI equivalent to 0.62, 1.46. The reduction in mortality was greatest in males, with the effect increasing with age. The HR of the male patients (over 75 years of age) was 0.59 at 95% confidence interval (CI) equivalent to 0.38, 0.87. Some evidence of the intervention being more effective in males in highly deprived groups were also observed. The largest reduction in the mortality rates was observed for males who belong to 50% least deprived groups, and those who were of 75 of years of age and above. Further, performance of the aforesaid system and method is established by determining (Number-Needed-to-Treat) NNT. The NNT provides a measurement of impact of the at least one healthcare intervention provided to the patients selected by the predictive model by estimating a number of patients need to be treated in order to save life of one person. Herein, for every 39 individuals identified through the predictive model and given the at least one healthcare intervention, 1 life was saved per year. For males of age 75 years and above, the NNT value was 9.

### BRIEF DESCRIPTION OF THE DRAWINGS

One or more embodiments of the invention will now be described, by way of example only, with reference to the following diagrams wherein:

FIGS. 1A and 1B are block diagrams of architectures of a system for assessing a risk of adverse medical events leading to unplanned healthcare and/or death, and for facilitating mitigation of the adverse medical events, in accordance with different embodiments of the present disclosure;

FIG. 2 is a simplified illustration of how data transformation is performed prior to risk scoring using a predictive model, in accordance with an embodiment of the present disclosure; and

FIGS. 3A and 3B are illustrations of steps of a method for assessing a risk of adverse medical events leading to unplanned healthcare and/or death, and for facilitating mitigation of the adverse medical events, in accordance with an embodiment of the present disclosure.

### DETAILED DESCRIPTION

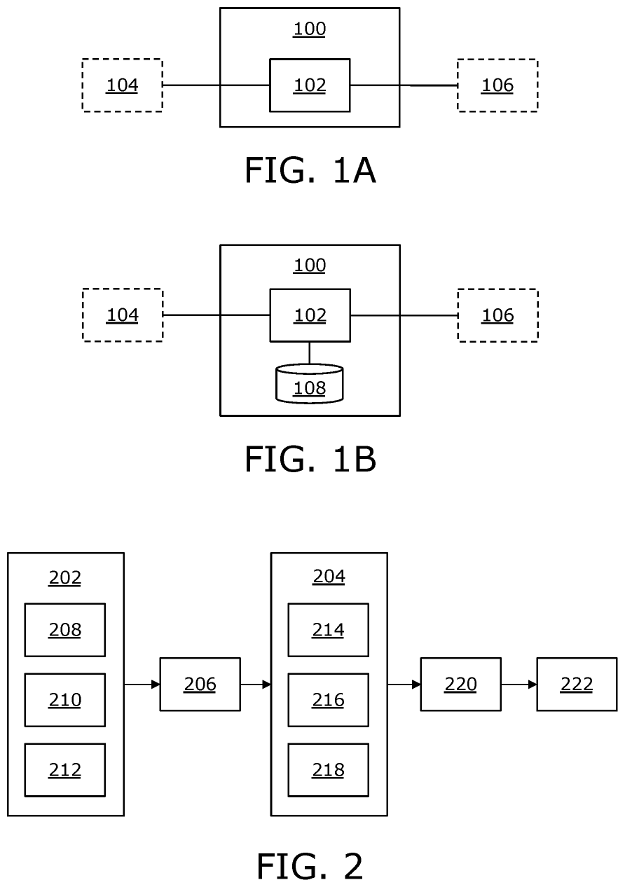

Referring to FIGS. 1A and 1B, illustrated are block diagrams of architectures of a system for assessing a risk of adverse medical events leading to unplanned healthcare and/or death, and for facilitating mitigation of the adverse medical events, in accordance with different embodiments of the present disclosure. The system 100 comprises at least one processor (depicted as a processor 102). The processor 102 is communicably coupled to at least the at least one data source (depicted as a data source 104). The processor 102 may also be communicably coupled to at least one first device (depicted as a first device 106) associated with a healthcare professional. In FIG. 1B, the system 100 is shown to further comprise a data repository 108 communicably coupled to the processor 102.

FIGS. 1A, and 1B are merely examples, which should not unduly limit the scope of the claims herein. A person skilled in the art will recognize many variations, alternatives, and modifications of embodiments of the present disclosure.

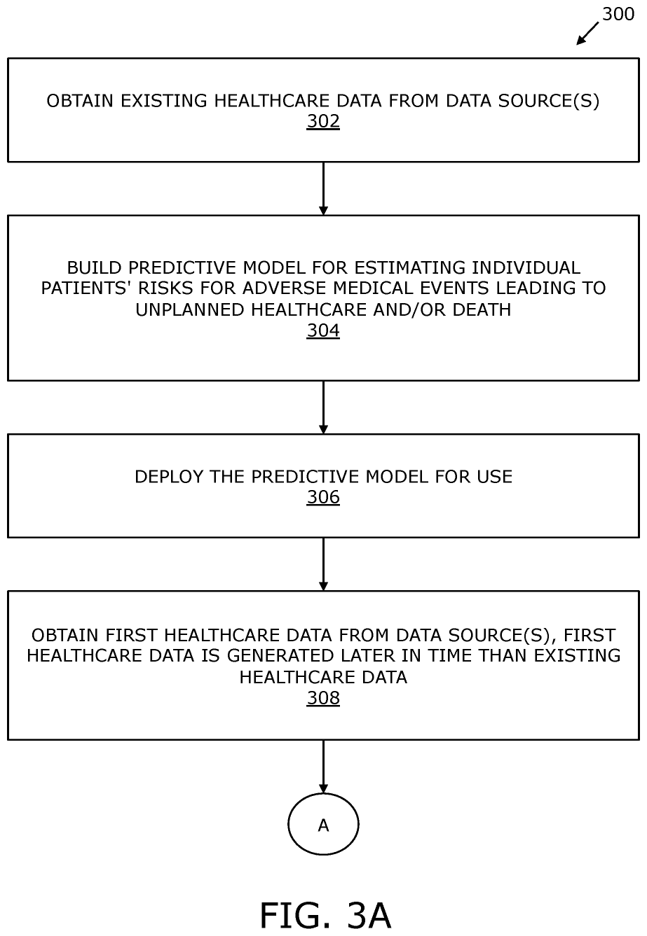

Referring to FIG. 2, illustrated is a simplified illustration of how data transformation is performed prior to risk scoring using a predictive model, in accordance with an embodiment of the present disclosure. A given healthcare data 202 is normalized into a unified feature set 204 using a feature normalization layer 206. the given healthcare data 202 includes accident and emergency data 208, inpatient data 210, and outpatient data 212. The given healthcare data 202 may also include prescription data, other medical data, information from medical databases, laboratory test results, primary care data, and similar. The unified feature set 204 comprises features pertaining to patient characteristics 214, medical diagnosis 216, and healthcare provider activity 218. Then, at 220, data quality and imputation checks are executed on the unified feature set. This yields a final feature set 222 which is ready for risk scoring using the predictive model.

FIG. 2 is merely an example, which should not unduly limit the scope of the claims herein. A person skilled in the art will recognize many variations, alternatives, and modifications of embodiments of the present disclosure.

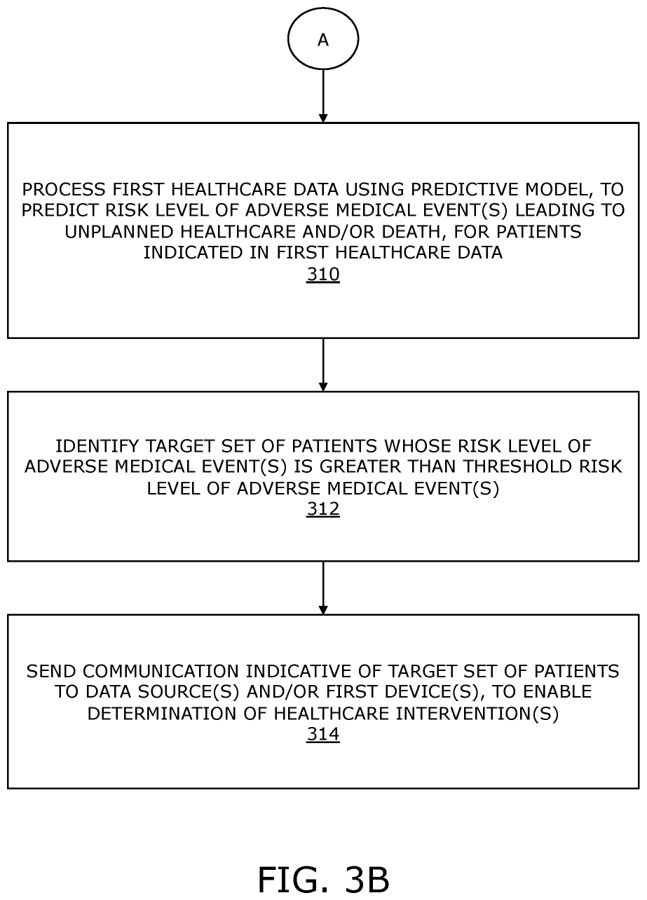

Referring to FIGS. 3A and 3B, illustrated are steps of a method 300 for assessing a risk of adverse medical events leading to unplanned healthcare and/or death, and for facilitating mitigation of the adverse medical events, in accordance with an embodiment of the present disclosure. At step 302, existing healthcare data is obtained from at least one data source, wherein the existing healthcare data comprises existing accident and emergency data, existing inpatient data, and existing outpatient data. At step 304, a predictive model is built for estimating individual patients'risks for adverse medical events leading to unplanned healthcare and/or death, using the existing healthcare data and a pre-defined set of medical conditions and pre-defined patient characteristics that are likely to lead to the adverse medical events. At step 306, the predictive model is deployed for use. At step 308, first healthcare data is obtained from the at least one data source, wherein the first healthcare data comprises first accident and emergency data, first inpatient data, and first outpatient data, the first healthcare data being generated later in time than the existing healthcare data. At step 310, the first healthcare data is processed using the predictive model, for predicting a risk level of at least one adverse medical event leading to unplanned healthcare and/or death, for each patient amongst a plurality of patients indicated in the first healthcare data. At step 312, a target set of patients is identified, wherein each patient belonging to the target set is one whose risk level of the at least one adverse medical event is greater than a threshold risk level of the at least one adverse medical event. At step 314, a communication indicative of the target set of patients is sent to the at least one data source and/or at least one first device associated with at least one healthcare professional, for enabling determination of at least one healthcare intervention. The at least one healthcare intervention, when provided to said patient, facilitates in at least partially mitigating the at least one adverse medical event which in turn reduces mortality of said patient.

The system has thus provided a diagnosis of a current health state and consequences like adverse medical events, enabling a determination of a healthcare intervention, i.e. the treatment of the current health state, which leads to an improved health of the patient(s) or at least an end to the degradation or mitigation of the health of the patient(s).

The aforementioned steps are only illustrative and other alternatives can also be provided where one or more steps are added, one or more steps are removed, or one or more steps are provided in a different sequence without departing from the scope of the claims herein.

1.-16. (canceled)

17. A system for assessing a risk of adverse medical events leading to unplanned healthcare and/or death, and for facilitating mitigation of the adverse medical events, the system comprising at least one processor configured to:
obtain existing healthcare data from at least one data source, wherein the existing healthcare data comprises existing accident and emergency data, existing inpatient data, and existing outpatient data;
build a predictive model for estimating individual patients'risks for adverse medical events leading to unplanned healthcare and/or death, using the existing healthcare data and a pre-defined set of medical conditions and pre-defined patient characteristics that are likely to lead to the adverse medical events;
deploy the predictive model for use;
obtain first healthcare data from the at least one data source, wherein the first healthcare data comprises first accident and emergency data, first inpatient data, and first outpatient data, the first healthcare data being generated later in time than the existing healthcare data;
process the first healthcare data using the predictive model, to predict a risk level of at least one adverse medical event leading to unplanned healthcare and/or death, for each patient amongst a plurality of patients indicated in the first healthcare data;
identify a target set of patients, wherein each patient belonging to the target set is one whose risk level of the at least one adverse medical event is greater than a first threshold risk level of the at least one adverse medical event;
send a communication indicative of the target set of patients to the at least one data source and/or at least one first device associated with at least one healthcare professional, for enabling determination of at least one healthcare intervention, wherein the at least one healthcare intervention, when provided to said patient, facilitates in at least partially mitigating the at least one adverse medical event which in turn reduces mortality of said patient.

18. The system according to claim 17, wherein the at least one first device is configured to receive a plurality of first inputs provided by the at least one healthcare professional, the plurality of first inputs pertaining to a selection of a first subset of patients from amongst the target set such that a portion of the first healthcare data that is associated with each patient selected to belong to the first subset complies with at least one inclusion criteria.

19. The system according to claim 18, wherein the at least one first device is further configured to receive a plurality of second inputs provided by the at least one healthcare professional, the plurality of second inputs pertaining to a selection of a second subset of patients from amongst the first subset of patients such that the portion of the first healthcare data that is associated with each patient selected to belong to the second subset is non-compliant with at least one exclusion criteria.

20. The system according to claim 17, wherein when building the predictive model, the at least one processor is configured to:
normalize the existing healthcare data into a unified feature set, wherein the unified feature set comprises features pertaining to patient characteristics, medical diagnosis, and healthcare provider activity;
execute data quality and imputation checks on the unified feature set;
train and validate the predictive model using a first portion of the unified feature set and at least one machine learning algorithm to build weights against each medical condition in the pre-defined set of medical conditions; and
test the predictive model that is trained, using a second portion of the unified feature set.

21. The system according to claim 20, wherein the predictive model is trained and validated using k-fold cross validation, and wherein prior to testing the predictive model that is trained, the at least one processor is further configured to:
generate model evaluation scores for the k folds; and
fine tune the predictive model by adjusting the weights, based on the model evaluation scores, for improving an accuracy of the predictive model.

22. The system according to any of claim 20, wherein the weights are built against each medical condition in the pre-defined set of medical conditions using pre-defined weights.

23. The system according to any of claim 20, wherein the patient characteristics comprise one or more of: age, gender, ethnicity, social and economic deprivation, and wherein the at least one processor is further configured to:
identify a set of vulnerable patients, based on the patient characteristics, wherein a vulnerable patient is one who belongs to one or more of: a vulnerable age group, a vulnerable gender, a vulnerable ethnicity, a deprived group;
determine a vulnerability score for each patient in the set of vulnerable patients, based on data associated with patient characteristics of said patient; and
enhance the risk level of the at least one adverse medical event for each vulnerable patient having a vulnerability score higher than a threshold vulnerability score, by a predetermined level.

24. The system according to claim 17, wherein the at least one processor is further configured to process the existing healthcare data to extract an additional feature set, wherein the additional feature set comprises time-dependent variables indicative of patient condition and activity, wherein when training the predictive model, the at least one processor is configured to also use the additional feature set.

25. The system according to claim 17, wherein the at least one processor is further configured to:
receive, from the at least one data source and/or the at least one first device, feedback pertaining to accuracy of the target set of patients and suitability of the target set of patients to receive the at least one healthcare intervention;
determine a performance metric of the predictive model, based on the feedback; and
initiate re-training of the predictive model based on the feedback and the performance metric.

26. The system according to claim 17, wherein the at least one processor is further configured to receive, for each patient belonging to the target set, an input indicative of the at least one healthcare intervention, from the at least one data source and/or the at least one first device.

27. The system according to claim 17, wherein the at least one processor is further configured to:
obtain second healthcare data from the at least one data source, wherein the second healthcare data comprises second accident and emergency data, second inpatient data, and second outpatient data, the second healthcare data being generated later in time than the first healthcare data;
process the second healthcare data to predict an updated risk level of the at least one adverse medical event for: each patient not belonging to the target set, each patient belonging to the target set who received the at least one healthcare intervention;
update the target set of patients, based on the updated risk levels; and
send a communication indicative of the updated target set of patients, to the at least one data source and/or the at least one first device.

28. A system according to claim 17, wherein the at least one processor is further configured to:
deploy a pseudonymization engine at the at least one data source, wherein the pseudonymization engine employs a hashing algorithm to pseudonymize a given healthcare data; and
deploy communication interfaces connecting the at least one data source with at least the at least one processor and the at least one first device to enable at least one of: sending of real-time updates of the given healthcare data, sending of the given healthcare data only upon pseudonymization, re-identification of the given healthcare data of a subset of patients within the target set of patients at a target device wherein the subset of patients includes patients with risk levels greater than a threshold high-risk level of the at least one adverse medical event.

29. The system according to claim 17, wherein the at least one data source is at least one of: a device associated with a healthcare facility, a device associated with a primary care provider, an out of hours (OOH) service, a device associated with a health trust, an ambulance service, a device associated with a mental health and community facility.

30. The system according to claim 17, wherein the at least one adverse medical event is at least one of: unplanned hospitalization, unplanned outpatient visit, requirement of emergency services, readmission to a healthcare facility, stranding in a healthcare facility, serious fall, frailty progression, worsening of existing medical conditions, emergence of new medical conditions, pediatric response exacerbation, high-intensity primary care usage.

31. A method for assessing a risk of adverse medical events leading to unplanned healthcare and/or death, and for facilitating mitigation of the adverse medical events, the method comprising:
obtaining existing healthcare data from at least one data source, wherein the existing healthcare data comprises existing accident and emergency data, existing inpatient data, and existing outpatient data;
building a predictive model for estimating individual patients'risks for adverse medical events leading to unplanned healthcare and/or death, using the existing healthcare data and a pre-defined set of medical conditions and pre-defined patient characteristics that are likely to lead to the adverse medical events;
deploying the predictive model for use;
obtaining first healthcare data from the at least one data source, wherein the first healthcare data comprises first accident and emergency data, first inpatient data, and first outpatient data, the first healthcare data being generated later in time than the existing healthcare data;
processing the first healthcare data using the predictive model, for predicting a risk level of at least one adverse medical event leading to unplanned healthcare and/or death, for each patient amongst a plurality of patients indicated in the first healthcare data;
identifying a target set of patients, wherein each patient belonging to the target set is one whose risk level of the at least one adverse medical event is greater than a threshold risk level of the at least one adverse medical event; and
sending a communication indicative of the target set of patients to the at least one data source and/or at least one first device associated with at least one healthcare professional, for enabling determination of at least one healthcare intervention, wherein the at least one healthcare intervention, when provided to said patient, facilitates in at least partially mitigating the at least one adverse medical event which in turn reduces mortality of said patient.

32. A computer program product comprising a non-transitory machine-readable data storage medium having stored thereon program instructions that, when accessed by at least one processor, cause the at least one processor to implement the method of claim 31.
